(19) United States
(12) Patent Application Publication
(10) Pub. No.: US 2023/0263583 A1
(43) Pub. Date: Aug. 24, 2023

(54) GEARED INSTRUMENTS

(71) Applicant: CMR Surgical Limited, Cambridge (GB)

(72) Inventors: Patrick Thornycroft, Cambridge (GB); Pedro Riera Martinez, Cambridge (GB)

(21) Appl. No.: 18/005,450

(22) PCT Filed: Jul. 13, 2021
(86) PCT No.: PCT/GB2021/051786

§ 371 Date: Jan. 13, 2023

(30) Foreign Application Priority Data
Jul. 14, 2020 (GB) 2010830.4

(51) Int. Cl. A61B 34/30; B25J 9/10

(52) U.S. Cl. CPC A61B 34/30; B25J 9/104; B25J 9/1035; A61B 2034/715

(57) ABSTRACT

A robotic surgical instrument comprising a shaft, an articulation attached to a distal end of the shaft, the articulation configured to articulate an end effector, the articulation driveable by a distal driving element, a driving mechanism comprising an instrument interface element secured to an end of a proximal driving element and configured to engage a drive interface element of a drive assembly, wherein motion of the drive interface element results in a first displacement of the end of the proximal driving element and a gearing mechanism engaging the proximal driving element and the distal driving element and being configured to transfer the first displacement of the end of the proximal driving element to a different second displacement of an end of the distal driving element.

### BACKGROUND

It is known to use robots for assisting and performing surgery. FIGS. 1 and 2 show a typical surgical robot 100 which comprises a base 101, an arm 102 and an instrument 103. The base supports the robot, and is itself attached rigidly to, for example, the operating theatre floor, the operating theatre ceiling or a trolley. The arm 102 extends between the base and the instrument. The arm is articulated by means of multiple flexible joints 104 along its length, which are used to position the surgical instrument 103 in a desired location relative to the patient. The surgical instrument is attached to the distal end of the robot arm. The surgical instrument penetrates the body of a patient at a port so as to access the surgical site.

A typical surgical instrument 103 shown in FIG. 3 comprises an instrument interface 301 by means of which the surgical instrument connects to the robot arm 102. A shaft 302 extends between the instrument interface 301 and an articulation 303. The articulation terminates in an end effector 304 and permits the end effector to move relative to the shaft 302.

It is desirable to develop a surgical robot able to control an attachable surgical instrument such that the end effector of the surgical instrument can be positioned in the desired location relative to a patient and be actuated so as to perform the desired surgical procedure.

### SUMMARY OF THE INVENTION

According to the first embodiment of the invention, there is provided a robotic surgical instrument as set out in the accompanying claims.

### BRIEF DESCRIPTION OF THE FIGURES

FIG. 1 shows a surgical robot and a patient.

FIG. 2 shows a surgical robot and associated control system.

FIG. 3 shows an instrument.

FIG. 4 shows an attachment of a robot arm for interfacing with an instrument.

FIGS. 5a and 5b show a more detailed view of an instrument.

FIG. 6 shows an interface of an instrument.

FIG. 7 shows a driving mechanism of an instrument.

FIG. 8 shows two views of a driving mechanism of an instrument, the driving mechanism comprising a gearing mechanism. The gearing mechanism comprises two pulleys and two driving elements.

FIG. 9 shows a gearing mechanism comprising a toothed belt and a toothed gear.

FIG. 10 shows a gearing mechanism comprising a toothed rack and a toothed gear.

FIG. 11 shows a gearing mechanism comprising at three straight rods, a toothed rack and a toothed gear.

FIG. 12 shows a gearing mechanism comprising one straight rod and one hook shaped rod, a toothed rack and a toothed gear

FIG. 13 shows a gearing mechanism comprising a push rod.

FIG. 14 shows a gearing mechanism comprising more than two pulleys.

FIG. 15 shows a gearing mechanism comprising two truncated cones.

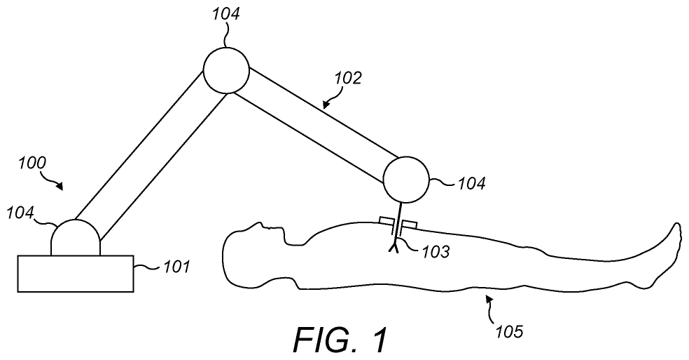

### DETAILED DESCRIPTION

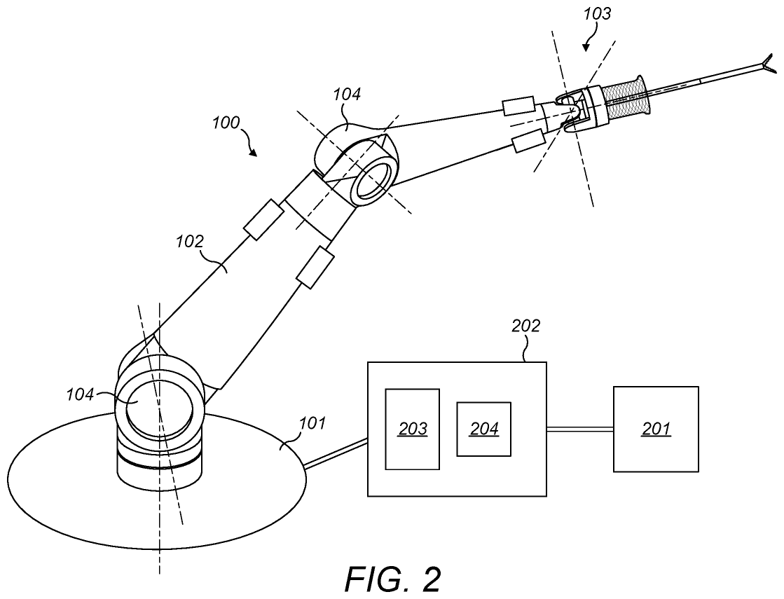

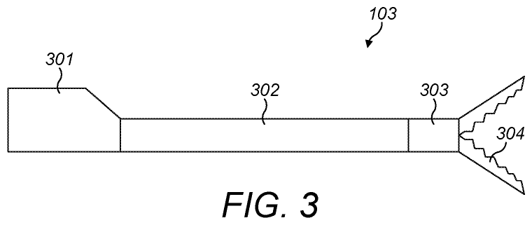

The following describes a robot comprising a robot arm and an instrument. The arm is generally of the form seen in FIG. 2. The instrument is generally of the form seen in FIG. 3.

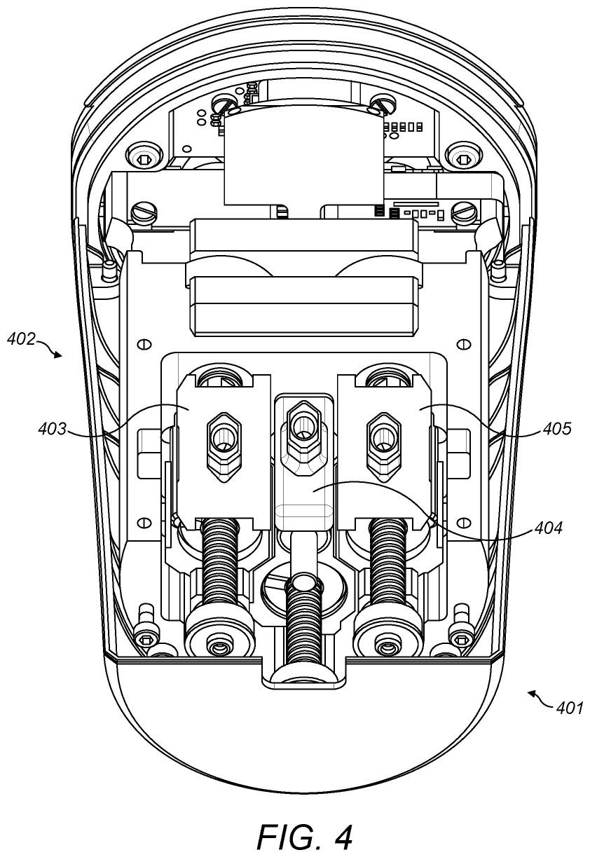

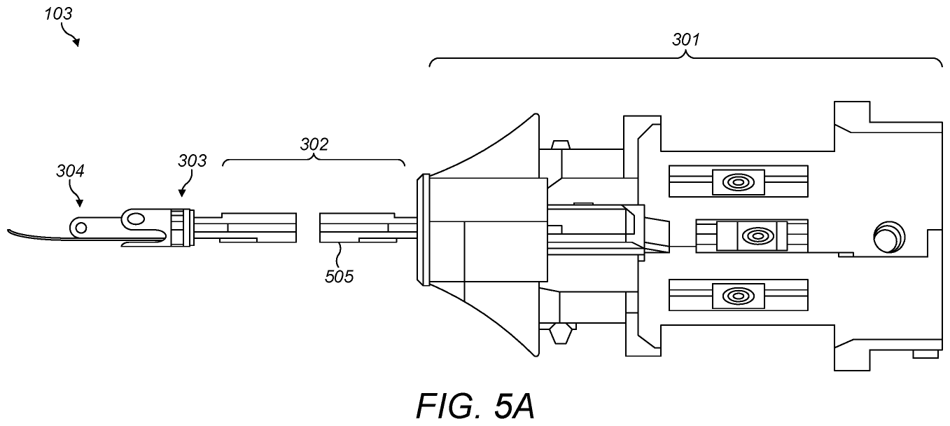

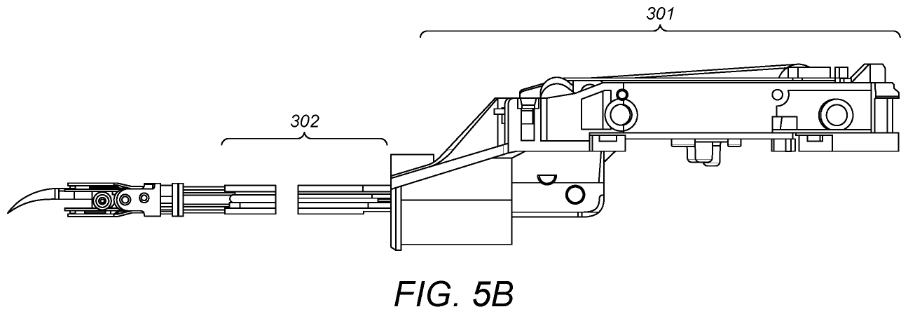

The arm 102 of the robot seen in FIG. 2 terminates in an attachment 401 for interfacing with the instrument, which is seen in FIG. 4. The attachment comprises a drive assembly 402 for driving articulation of the instrument 103. The drive assembly interface 402 interfaces with the instrument interface 301 seen in FIGS. 3, 5a and 5b. Moveable interface elements 403, 404, 405 of the drive assembly interface engage corresponding moveable interface elements of the instrument interface in order to transfer drive from the robot arm 102 to the instrument 103.

The instrument 103 is shown in more detail in FIGS. 5a and 5b and comprises an end effector 304 for performing an operation. The end effector may be smooth jaws, serrated jaws, a gripper, a pair of shears, needle graspers, biopsy needles or needles for injecting drugs. The instrument comprises an articulation 303 between the instrument shaft 302 and the end effector 304. The articulation comprises several joints which permit the end effector to move relative to the shaft of the instrument. The joints in the articulation are actuated by driving elements 505, such as cables.

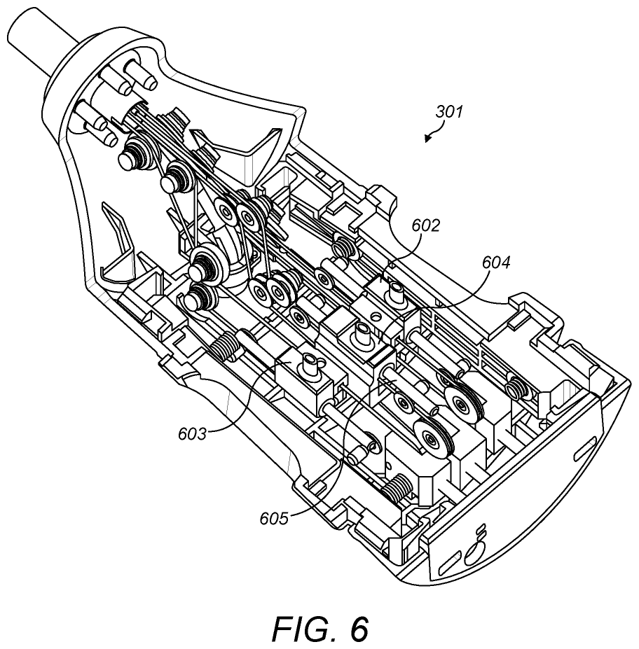

At the distal end of the instrument shaft 302, the driving elements 505 are connected to the end effector 304 and are used to actuate the joints in the articulation. At the proximal end of the shaft, the driving elements are secured to interface elements of the instrument interface 301. FIG. 6 illustrates an instrument interface 301 comprising instrument interface elements 602, 603, 60. In this instrument interface, each instrument interface element is secured to a driving element e.g. instrument interface element 604 is secured to driving element 605.

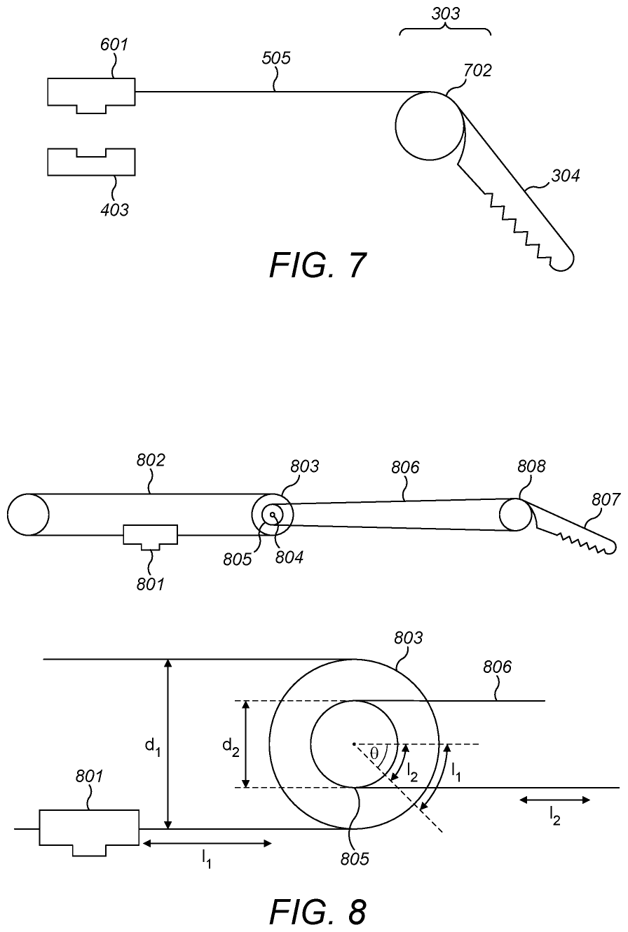

FIG. 7 is a schematic of the driving mechanism of an instrument interface of an instrument. The driving mechanism is preferably located at the proximal end of the instrument shaft, but may be located at any point between the proximal end of the instrument shaft and the end effector. A drive assembly interface element 403 (of a robot arm) engages with the instrument interface element 601 of an instrument. In FIG. 7, the driving element 505 is secured at one end to the instrument interface element, and at the other end to the end effector 304 via a joint 702. The joint forms part of the articulation 303. The drive assembly interface element 403 engages with the instrument interface element 601 such that motion of the drive assembly interface element is transferred to motion of the instrument interface element. The instrument interface element is secured to the driving element such that motion of the instrument interface element 403 is transferred to motion of the driving element 505. Since the driving element is also secured to the end effector 304, motion of the instrument interface element is directly transferred to motion of the end effector. Thus, motion of the drive assembly interface element results in motion of the end effector. The robot arm 102 transfers drive to the end effector 304 of the instrument 103 as follows: movement of a drive assembly interface element 403 moves an instrument interface element 601 which moves a driving element 505 which moves a joint 702 of the articulation 303 which moves the end effector 304. In such a driving mechanism, movement of the drive interface element is transferred to movement of the end effector as a function of a fixed set of parameters (the length of the driving element, the friction of the joint etc.) In this way, the ratio of motion of the drive interface element to motion of the end effector is fixed. i.e. for an instrument with this driving mechanism, the ratio of motion of the drive interface element to motion of the end effector is always the same. In some cases, this ratio is 1:1. In many examples, the displacement of the driving element is equal to the displacement of the end effector. In other examples the force exerted by the drive assembly interface element on the instrument interface element is equal to the force exerted by the end effector.

FIG. 8 illustrates a first example of a driving mechanism of an instrument. The driving mechanism comprises a gearing mechanism located between an instrument interface element and an end effector. In this example, the gearing mechanism comprises a first pulley 803 and a second pulley 805, two pulleys being disposed about a single axis 809.

The driving mechanism comprises at least one instrument interface element 801. The instrument interface element is secured to a proximal driving element 802. The proximal driving element 802 seen in FIG. 8 is a single length of cable. Each end of the proximal driving element is secured to an end of the instrument interface element. The proximal driving element is looped around a first pulley 803. The first pulley 803 rotates about an axis 809. A second pulley 805 rotates about the axis 809 of the first pulley 803. FIG. 8 shows that first pulley 803 has a diameter that is larger than the diameter of second pulley 805. The first and second pulleys 803 and 805 form part of the gearing mechanism located between the instrument interface element 801 and the end effector 807. A distal driving element 806 is looped around the second pulley 805 and is formed of a single loop of cable. The proximal driving element 802 extends away from the axis 809 in a first direction. The distal driving element extends away from the axis 809 in a second direction. In the example seen in FIG. 8, the proximal driving element 802 and the distal driving element 806 extend away from the axis 809 in opposing directions. In this way, the axis of the first and second pulleys 809 is located generally in between the proximal driving element and the distal driving element. The first and second pulleys may be disposed about a common axle 804, coincident with axis 809. The distal driving element 806 is secured to a joint 808. The joint 808 is fixedly attached to an end effector 807. The first and second pulleys 803 and 805 form part of the gearing mechanism, which may be positioned anywhere between the instrument interface element and the joint.

In one example, the gearing mechanism is positioned closer to instrument's proximal end than its distal end. In this arrangement, when the instrument is used on a patient and inserted into a surgical port, the gearing mechanism stays outside of the patient. During a surgical procedure, this is beneficial as a practitioner is able to make adjustments to the gearing mechanism, for example a surgeon may wish to disengage the proximal and distal driving elements in order to change the end effector. Such an arrangement also allows for the first and second pulleys to have diameters larger than the maximum size of a surgical port in the patient. Furthermore, placing the gearing mechanism near the proximal end of the instrument also reduces the torque needed to alter the position of the instrument.

The instrument interface element is configured to engage with a drive assembly interface element of a robot arm (not shown). The instrument interface element can be driven by the drive assembly interface element such that motion of the drive assembly interface element results in motion of the instrument interface element.

As previously mentioned, the instrument interface element 801 is secured to the proximal driving element 802. The proximal driving element is constrained to move about the first pulley. The first pulley 803 can rotate about its axis 809. At least a point on the proximal driving element 802 is secured to the pulley 803 such that movement of the proximal driving element around the pulley results in rotation of the pulley about its axis. For example, a bead may be used to secure the driving element to the pulley. The first pulley may be secured to axle 804 which is also rotatable about the axis such that rotation of the first pulley results in rotation of the axle. For example, the axle may be in the form of a sheath which is rotatable around the axis 809 of the pulley.

FIG. 8 shows the second pulley 805 which can rotate about the axis 809 of the first pulley 803. The distal driving element 806 is constrained to move about the second pulley. At least a point on the distal driving element is secured to the second pulley. The first and second pulleys may be disposed about and fixed to the axle 804. In such an example, rotation of the first pulley about its axis, is transferred, via the axle, to rotation of the second pulley. The distal driving element 806 is secured to the second pulley 805 such that rotation of the second pulley results in movement of the distal driving element. For example, a bead may be used to secure the driving element to the pulley. The distal driving element is secured to the end effector 807 about a joint 808. Movement of the distal driving element is therefore transferred to movement of the end effector. In other examples, the first and second pulleys may be secured directly to one another, or rotation of one pulley may be transferred to rotation of the other pulley using a different mechanism.

Therefore, in this example, a robot arm (not shown) transfers drive to the end effector 807 of the instrument as follows: movement of a drive assembly interface element (not shown) moves an instrument interface element 801 which moves a driving element 802 which moves a first pulley 803, which moves a second pulley 805, which moves a driving element 806, which moves joint 808 which moves the end effector 807. In the example shown, movement of an instrument interface element results in movement of a pulley because the driving element is secured to the pulley. However, in other examples, the driving element may not be fixed to the pulley. In such an example, movement of the pulley may be achieved due to a high coefficient of friction between the driving element and the pulley. One or both of the driving element and pulley may comprise a raised profile and/or grooves which contribute to the high coefficient of friction between the components.

In FIG. 8, the driving elements are cables. However, in other examples, the driving elements could be any other elongate elements. For example, the driving elements could be chains, belts, racks or rods. Each driving element could be a single length, a loop or alternatively could be formed a number of parts, for example a pair of driving elements. In the example of FIG. 8, the driving elements are flexible, but may alternatively be rigid. The driving elements could comprise rigid and flexible portions. The proximal and distal driving elements may be different types of driving element. Each driving element may be formed of a material such that it elongates according to a known model (e.g. spring/damper model) when force is applied to it.

The diameter of the first pulley 803 is different to the diameter of the second pulley 805. In the enlarged version of the gearing mechanism shown in FIG. 8, the diameter of the first pulley d1 is greater than the diameter of the second pulley d2. In this example, the proximal driving element is driven linearly, i.e. linear motion of the drive assembly interface element results in linear motion of the instrument interface element which results in linear motion of the proximal driving element. However, in other examples, the proximal driving element may be driven rotationally. When the instrument interface element is driven (either linearly or rotationally), the end of the proximal driving element attached to the instrument interface element is displaced by a first displacement l1. First displacement l1 is transferred to rotation of the first pulley, causing the first pulley 803 to rotate by an angle θ. Equation 1 illustrates the relationship between linear displacement l1 and rotational displacement θ.

$$l 1 = π d 1 \times \frac{θ}{360} \qquad ( Equation 1 )$$

As explained above, the rotation of the first pulley is transferred to rotation of the second pulley. The second pulley is thus also rotated by angle θ. The distal driving element is secured to the second pulley such that rotation of the second pulley results in displacement of an end of the distal driving element. Specifically, the end of the distal driving element which is attached to the end effector is displaced by a second displacement l2. Equation 2 illustrates the relationship between linear displacement l2 and rotational displacement θ.

$$l 2 = π d 2 \times \frac{θ}{360} \qquad ( Equation 2 )$$

The displacement of the distal driving element l2 can therefore be calculated using the following equation, where d1 is the diameter of the first pulley and d2 is the diameter of the second pulley.

$$l 2 = \frac{l 1 \times d 2}{d 1} \qquad ( Equation 3 )$$

The ratio of the first and second displacements is therefore equal to the ratio of the diameter of the first pulley to the diameter of the second pulley

$$( \frac{l 1}{l 2} = \frac{d 1}{d 2} ) .$$

Thus provided d1≠d2, l1≠l2. In this way, the proportion of the displacement of an end of the proximal driving element which is transferred to displacement of an end of the distal driving element can be altered by changing the ratio of the diameter of the first pulley to the diameter of the second pulley.

The force exerted on the proximal driving element 802 by driving element 801 so that the end of the proximal driving element is displaced by displacement l1 is F1. When the end of the distal driving element is displaced by displacement l2, the force exerted on the end effector by the distal driving element and thus by the end effector is F2. Neglecting inefficiencies caused by, for example friction, displacement l1 is inversely proportional to force F1 and displacement l2 is inversely proportional to force F2. Therefore, the ratio of the first displacement l1 to the second displacement l2 is the inverse of the ratio of F1 to F1. Accordingly,

$$\frac{F 1}{F 2} = \frac{d 2}{d 1} \qquad ( Equation 4 )$$

The requirements of an end effector vary significantly across different types of surgical procedures. For example, in some surgical procedures, the force required to be imparted by the end effector may be less than that commonly applied by the drive assembly. It may therefore be desirable to reduce the sensitivity of the end effector of the instrument. One way to achieve this is to reduce the proportion of motion of the instrument interface element that is transferred to movement of the end effector. i.e. ensure that l2<l1. In this example, it may be beneficial for the diameter of the first pulley to be greater than the diameter of the second pulley. Equation 5 illustrates (with reference to equation 3) the effect on the respective displacements when the diameter of the first pulley is greater than the diameter of the second pulley.

$$\begin{matrix}d 1 > d 2 & \frac{d 2}{d 1} < 1 & l 2 < l 1\end{matrix} \qquad ( Equation 5 )$$

In another example, where the end effector is a pair of grippers and is required to grip a needle, the end effector may be required to apply high forces for a prolonged period of time. In other words, it is desirable to maximise the force output by the end effector. In this example, it would therefore be preferable to increase the proportion of motion of the instrument interface element that is transferred to motion of the end effector. In this example, it could be beneficial for the diameter of the second pulley to be greater than the diameter of the first pulley. The term “proportion” is not intended to mean less than one. It will be clear that the gearing mechanism shown in FIG. 8 can be implemented in a way so as to allow the displacement of the distal driving element to be greater than the displacement of the proximal driving element. In other words, a proportion of motion can be transferred, where the proportion is greater than one. Equation 6 illustrates (with reference to equation 3) the effect on the respective displacements when the diameter of the second pulley is greater than the diameter of the first pulley.

$$\begin{matrix}d 2 > d 1 & \frac{d 2}{d 1} > 1 & l 2 > l 1\end{matrix} \qquad ( Equation 6 )$$

The gearing mechanism can thus be used to mechanically modulate the amount of motion of the instrument interface element (effected by the robot arm) which is transformed into motion of the end effector.

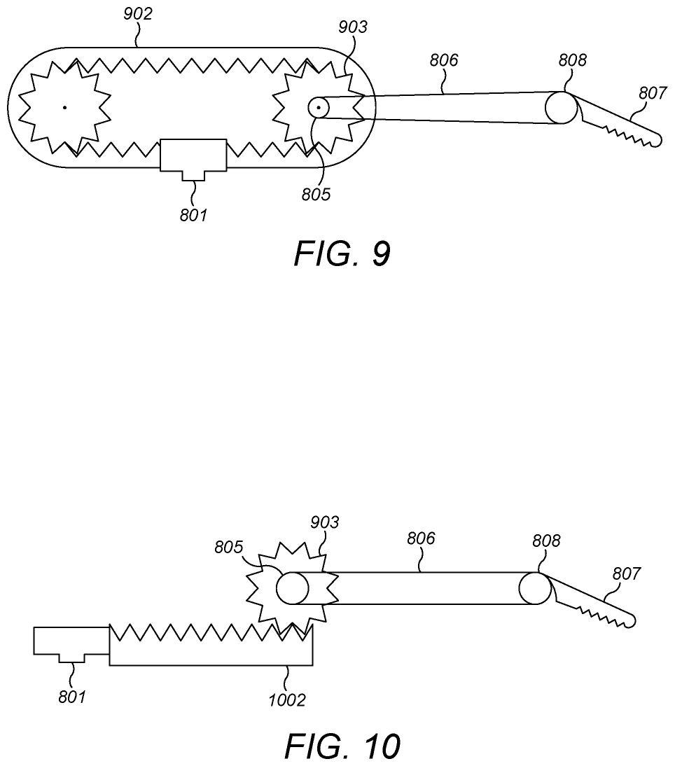

FIGS. 9, 10 and 11 illustrate further examples of gearing mechanisms.

FIG. 9 illustrates a second example of a driving mechanism. This driving mechanism comprises many of the same elements as the driving mechanism shown in FIG. 8. In contrast to the example seen in FIG. 8, the driving mechanism comprises a toothed belt and a toothed gear. The proximal driving element is a toothed belt 902. The toothed belt comprises teeth. The toothed belt 902 engages with the first pulley, which in this example is a toothed gear 903. The toothed gear also comprises teeth. The teeth of the toothed belt are configured to mesh with the teeth of the toothed gear. The toothed belt 902 engages with the toothed gear 903 in that the teeth of the toothed belt mesh with the teeth of the toothed gear. In this way, motion of the toothed belt is transferred to rotation of the toothed gear.

The driving mechanism operates in a similar way to that described with reference to FIG. 8. The second pulley 805 is disposed about the axis of the toothed gear 903. The second pulley and the toothed gear may be disposed about a common axle. Therefore, in this example, a robot arm (not shown) transfers drive to the end effector 807 of the instrument as follows: movement of a drive assembly interface element (not shown) moves an instrument interface element 801 which moves a toothed belt 902 which moves a toothed gear 903, which moves a second pulley 805, which moves a driving element 806, which moves joint 808 which moves the end effector 807.

Since the respective teeth of the driving element and the pulley are closely engaged with one another, slipping between the driving element and the pulley is less likely and thus motion can be transferred more efficiently.

FIG. 10 illustrates a third driving mechanism. In this example, the instrument interface element 801 is secured to a toothed rack 1002. The toothed rack is rigid. The toothed rack comprises teeth. The first pulley is a toothed gear 903. The toothed rack 1002 engages with the toothed gear 903. The teeth of the toothed belt are configured to mesh with the teeth of the toothed gear. The toothed belt 902 engages with the toothed gear 903 in that the teeth of the toothed belt mesh with the teeth of the toothed gear. Displacement of the instrument interface element is equal to displacement of the toothed rack. Displacement of the toothed rack is transferred to rotation of the toothed gear.

The driving mechanism operates in a similar way to that described with reference to FIGS. 8 and 9. The second pulley 805 is disposed about the axis of the toothed gear 903. The second pulley and the toothed gear may be disposed about a common axle. Therefore, in this example, a robot arm (not shown) transfers drive to the end effector 807 of the instrument as follows: movement of a drive assembly interface element (not shown) moves an instrument interface element 801 which moves a toothed rack 1002 which moves a toothed gear 903, which moves a second pulley 805, which moves a driving element 806, which moves joint 808 which moves the end effector 807.

In the FIGS. 9 and 10, only the proximal driving element and first pulley are shown as comprising teeth. However, the distal driving element and second pulley may also comprise teeth. Alternatively, only the distal driving element and second pulley may comprise teeth.

FIGS. 9 and 10 illustrate examples of driving mechanisms in which at least one driving element is rigid. Rigid driving elements are less prone to strain under tension than more flexible driving elements. Furthermore, rigid elements can undergo both pushing and pulling forces. Using rigid driving elements may in some cases reduce the amount of space which is taken up by the driving mechanism. FIGS. 9 and 10 illustrate examples in which the rigid driving elements are racks comprising teeth. Toothed racks such as these may be easily locked in a desired position as they are engageable at any point along their length.

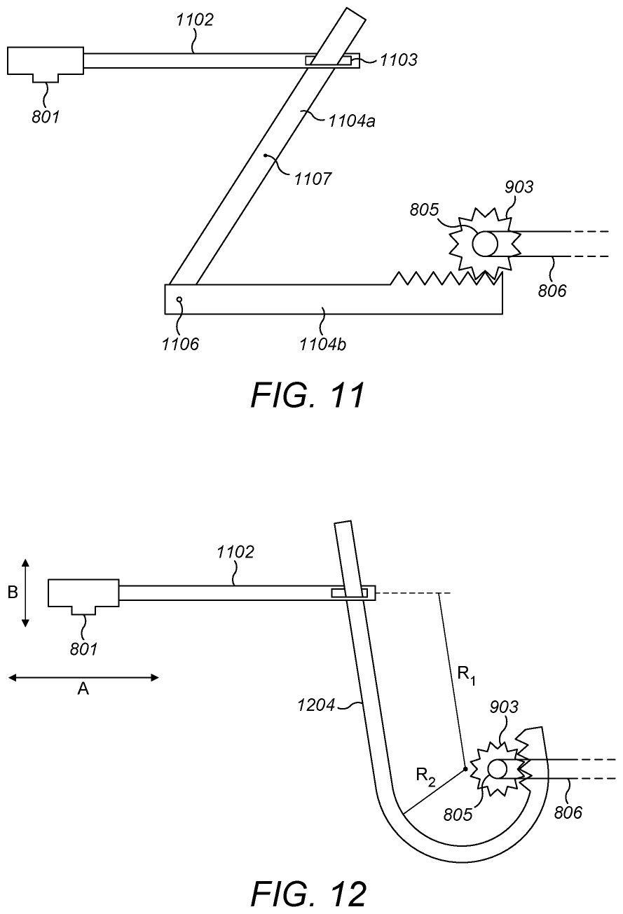

FIG. 11 shows a fourth example of a driving mechanism. In this example, the instrument interface element 801 is secured to an end of a first rod 1102. The rod 1102 is stiff and extends in a straight line. The rod extends away from the instrument interface element in a first direction, the first direction being the direction of translation of the instrument interface element. The rod 1102 comprises an aperture 1103 at an opposite end to the instrument interface element. The aperture may be in the form of a slot. A second rod 1104a is slotted through the aperture 1103. The second rod 1104a extends away from the aperture in a second direction at an angle to the first direction. The second rod 1104a is stiff and extends in a straight line. A third rod 1104b is pivotally mounted to the second rod 1104a about point 1106. Rod 1104b is also stiff and extends in a straight line. Pivot point 1106 is positioned at the opposite end of the second rod 1104a from the portion of the rod which passes through aperture 1103. The third rod 1104b extends away from pivot point 1106 in the first direction. Generally, rods 1102, 1104a and 1104b form a “Z” shape. In this example, at least a portion of the third rod 1104b comprises teeth. In the example shown, the end of the third rod 1104b which is distal from the first rod 1102 and second rod 1104a is a toothed rack. The gearing mechanism further comprises a toothed gear 903. The third rod 1104b engages with the toothed gear 903. Thus, in this example, the proximal driving element comprises three or more rods and the first pulley is a toothed gear. In other examples, the third rod 1104b may connect to the driving element 806 in another way.

In the example shown in FIG. 11, the rods 1104a and 1104b are configured to move relative to one another. Rods 1104a and 1104b are configured to pivot with respect to one another about pivot point 1106. The second rod 1104a is able to rotate about an axis 1107. In the example shown in FIG. 11, axis 1107 is positioned approximately halfway along second rod 1104a.

Therefore, in this example, a robot arm (not shown) transfers drive to the end effector 807 of the instrument as follows: movement of a drive assembly interface element (not shown) moves an instrument interface element 801 which moves a first rod 1102. Movement of the first rod 1102 moves the proximal end of the second rod 1104a, causing it to rotate about axis 1107. Because rods 1104a and 1104b are able to pivot relative to one another about point 1106, rotation of second rod 1104a moves third rod 1104b. Since the distal end of the third rod 1104b is a toothed rack engaged with a toothed gear 903, motion of the third rod 1104b causes rotation of toothed gear 903. As previously described with respect to FIGS. 8 to 10, this causes rotation of a second pulley 805, which moves a driving element 806, which moves joint 808 which moves the end effector 807.

In the example shown in FIG. 11, pivot point 1106 comprises a pin which passes through rods 1104a 1104b to join them whilst allowing them to rotate relative to one another. In a further example, pivot point 1106 may comprise an aperture in second rod 1104a (similar to aperture 1103) through which third rod 1104b is slotted. Alternatively, third rod 1104b may comprise an aperture through which second rod 1104a is slotted.

In another example, rods 1104a and 1104b are fixed relative to one another to form an L-shape. In this example, the resulting composite rod is not able to rotate about an axis. Translation of the first rod 1102 simply results in translation of the rods 1104a and 1104b. The remaining elements of the driving mechanism operate in the same manner as previously described.

In the example shown in FIG. 11, axis 1107 is positioned approximately halfway along rod 1104a. However, the arrangement may be configured such that the axis can be positioned at any point along the length of rod 1104a. By moving the position of the axis, the ratio of the displacement of first rod 1102 to the displacement of third rod 1104 can be adjusted.

Since the axis 1107 can take any position along the second rod 1107, this arrangement allows for fine tuning of the ratio between the force imparted on the drive interface element 801 F1 and the force imparted on the driving element 806 and thus the end effector, F2.

In a similar example shown in FIG. 12, the second and third rods may be replaced with a single hook-shaped rod 1204. The hooked rod comprises a straight portion at its proximal end and a curved portion at its distal end. As per the example shown in FIG. 11, the straight portion of the rod is joined to first rod 1102. A bearing connects the rods while allowing them to rotate relative to one another. As seen in FIG. 12, the hooked rod may pass through an aperture in the first rod. Alternatively, the bearing may be a pin passing through both rods. The curved portion of the rod comprises teeth and may engage a toothed gear in the same way as the arrangement of FIG. 11. The rod is curved so as to form a partial circle. In the example shown in FIG. 12, the teeth are positioned on the inside of the curved portion of the rod. When the teeth are located on the inside of the partial circle formed by the rod, the toothed gear is positioned within the partial circle. The partial circle formed by the curved portion of the rod has an effective radius R₂.

In this example, a robot arm transfers drive to the end effector of the instrument as follows: movement of a drive assembly interface element moves an instrument interface element 801 in a direction indicated by arrow A which moves a first rod 1102 in the same direction. Movement of the first rod moves the hooked rod 1204. Since the distal end of the hooked rod is a toothed rack engaged with a toothed gear, motion of the hooked rod causes rotation of toothed gear 903. This causes rotation of a second pulley 805, which moves a driving element 806, which moves a joint which moves an end effector (not shown).

FIG. 12 shows that the relative positions of the first rod 1102 and the hooked rod 1204 can be altered by moving the instrument interface element 801 and first rod 1102 in the directions indicated by arrows B. By altering the relative positions of the first rod 1102 and the hooked rod 1204, the distance between the first rod and the curved part of the hooked rod R₁ can be altered. For example, the arrangement may be altered such that the first rod engages the hooked rod closer to the curved portion of the hooked rod, thereby reducing the distance R₁. In this way, the force transmitted from the instrument interface element 801 to the distal driving element 806 and end effector (not shown) can be altered.

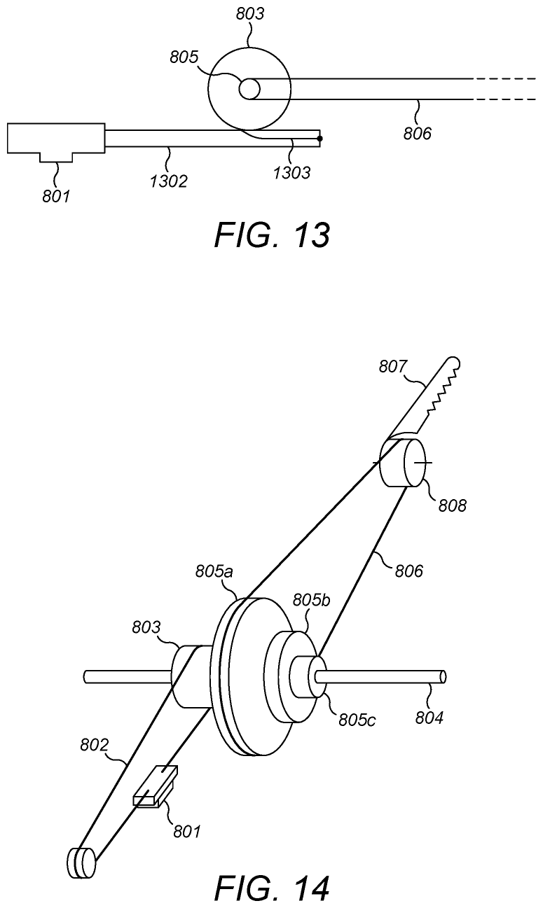

FIG. 13 illustrates a sixth example of a driving mechanism. In this example, the instrument interface element 801 is secured to a push rod 1302. The push rod is rigid. A driving element 1303 is secured to the push rod. In the example shown, the instrument interface element is secured to a proximal end of the push rod and the driving element is secured to an opposite distal end of the push rod. The driving element may be partially rigid. The driving element may be elastic. The driving element is constrained to move about a first pulley 803. The driving element 1303 wraps at least partially around pulley 803. In the example shown in FIG. 12, the driving element 1303 wraps around a portion of pulley closest to the proximal end of the push rod. Pulley 803 is positioned adjacent to the push rod and generally between the instrument interface element and the distal end of the push rod at which the driving element is secured. The driving element 1303 starts at the distal end of the push rod and follows a path partly along the length of the push rod towards the proximal end of the push rod (and the instrument interface element 801) before wrapping around pulley 803 in a direction first away from the push rod and then back towards the push rod's distal end. The push rod may comprise a groove and the driving element 1303 may follow the path partly along the length of the push rod within the groove. In the example shown in FIG. 12, pulley 803 is in contact with push rod 1302, but in other examples there may be a gap between the two components.

Motion of the instrument interface element 801 is transferred to motion of the push rod 1302. Since the driving element 1303 is secured to the push rod 1302, motion of the instrument interface element and push rod is transferred to motion of the driving element. As the driving element wraps around the pulley, motion of the driving element is transferred to rotation of the first pulley 803. As previously described, first pulley 803 is fixed to second pulley 805 such that rotation of the pulley 803 results in rotation of the pulley 805. Rotation of pulley 805 causes motion of distal driving element 806. Distal driving element 806 may be secured to an end effector about a joint such that movement of the driving element 806 is transferred to movement of the end effector. FIG. 13 shows distal driving element 806 being a loop of cable. However, in other examples, the driving element may be a single length of cable having one end secured to second pulley 805 and a second end (not shown) secured to an end effector.

In this example, a robot arm (not shown) transfers drive to the end effector 807 of the instrument as follows: movement of a drive assembly interface element (not shown) moves an instrument interface element 801 which moves a pushrod 1302. Motion of the pushrod 1302 moves the proximal driving element 1303, which moves a first pulley 803, which moves a second pulley 805, which moves a driving element 806, which moves joint 808 which moves the end effector 807.

FIG. 14 shows a driving mechanism similar to that seen in FIG. 8 but modified to include further pulleys. In contrast to the example seen in FIG. 8 which shows two pulleys 803 and 805 disposed about axle 804, FIG. 14 shows a driving mechanism comprising four pulleys disposed about axle 804. In this example, the proximal driving element 802 is constrained about pulley 803 and the distal driving element 806 is constrained to move about pulley 805a. Pulley 805a is rotationally fixed relative to pulley 803 such that when pulley 803 rotates, pulley 805a also rotates. Pulleys 805b and 805c are also positioned on axle 804 and configured to rotate about the axis of pulley 803. Pulleys 805b and 805c are also rotationally fixed relative to pulley 803 such that when pulley 803 rotates, pulleys 805b and 805c also rotate. Each of pulleys 803, 805a, 805b and 805c have a diameter. The diameter of each pulley is different to the diameter of each of the other pulleys. In this example, pulley 805c has a diameter that is smaller than first pulley 803, but the diameters of pulleys 805a and 805b are both larger than the diameter of pulley 803.

A robot arm (not shown) transfers drive to the end effector 807 of the instrument as described with reference to FIG. 8. In the example shown in FIG. 14, the position of the distal driving element 806 can be shifted so that it can be constrained to move about either pulley 805a, pulley 805b or pulley 805c. Since each of pulleys 805a, 805b and 805c have a different diameter, altering the pulley about which the distal driving element is constrained to move, alters the ratio of the diameter of the first pulley to the diameter of the second pulley. As explained above, the proportion of the displacement of an end of the proximal driving element 802 which is transferred to displacement of an end of the distal driving element 806 can be altered by changing the ratio of the diameter of the first pulley 803 to the diameter of the second pulley 805. Therefore, changing the pulley about which the distal driving element is constrained to move, alters the proportion of motion transferred from the drive assembly to the end effector. The driving mechanism may comprise a mechanism configured to allow the operator to change the pulley about which the distal driving element is constrained to move. For example, the driving mechanism may comprise a derailleur, further switches or levers.

Using this arrangement, the operator can choose a ratio of the diameter of the first pulley to the diameter of the second pulley from three discrete options. The operator can therefore select (from three options) the proportion of motion to be transferred from the instrument interface element to the end effector. In this way, the force output by the end effector from a single input at the drive assembly is adjustable. Therefore, a single instrument is customisable and can be adapted for a variety of surgical procedures. The ratio of the displacement of the end of the proximal driving element l1 to the displacement of the end of the distal driving element l2 can be altered so as to be closer to the desired ratio for a particular application. Furthermore, altering the ratio l1/l2 may also change the experience for the operator performing a procedure using the robot. For example, the instrument being more or less sensitive to input forces (driving signals) may affect the ease with which the operator can control the end effector. This gearing mechanism thus enables the operator to select a ratio so as to provide a different handling sensation while controlling the end effector to perform a surgical procedure.

In this example there are three second pulleys, each having a different diameter, and therefore three discrete options for the ratio d2/d1. In another example, there are only two second pulleys about which the distal driving element can be constrained to move. In another example, there are more than three second pulleys. In a further example, the position of the proximal driving element 802 can be shifted so that it can be constrained to move about any one of a number of pulleys. In this example, the distal driving element 806 may be constrained to move about a single pulley. Alternatively, both driving elements 802 and 806 can be constrained to move about any of a number of pulleys.

This modification has been illustrated using the example of a driving mechanism seen in FIG. 8. The same modification can be made to any of the driving mechanisms previously described. The mechanisms shown in any of FIGS. 9 to 13 may be modified such that multiple pulleys are disposed about the same axis.

The gearing mechanism shown in FIG. 14 may be positioned anywhere between the end effector 807 and instrument interface element 801. However, it is preferable that the gearing mechanism is positioned closer to the instrument's proximal end (the instrument interface element) than its distal end (the end effector). During a surgical procedure, in which the instrument is inserted into a port in the patient, when the gearing mechanism is positioned at the instrument's proximal end, it will not be inserted into the surgical port. Accordingly, the mechanism is more accessible to a practitioner such that the practitioner may adjust the gearing mechanism by hand. For example, in order to adjust the ratio between diameters of the first and second pulleys, a surgeon may disengage the distal driving element and re-position the distal driving element such that it is constrained by a different pulley.

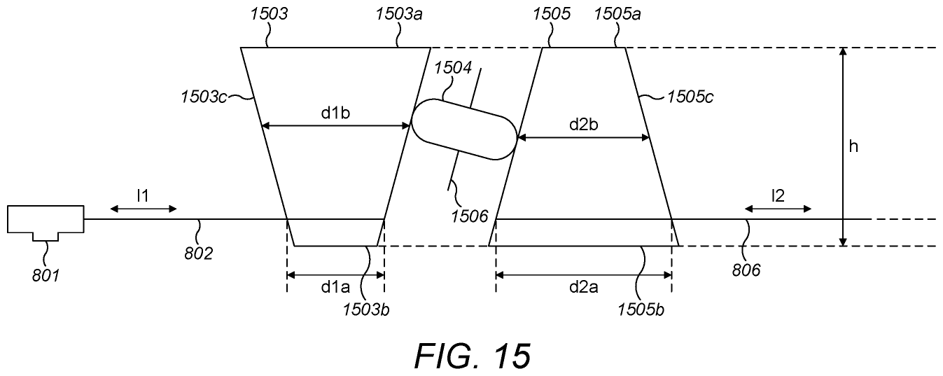

FIG. 15 illustrates a further example of a driving mechanism, where the driving mechanism comprises two truncated cones. The instrument interface element 801 is secured to a proximal driving element 802. The proximal driving element 802 engages a first truncated cone 1503. The first truncated cone has two planar circular faces 1503a and 1503b which are parallel to one another and one curved face 1503c. Circular face 1503a has a diameter greater than that of circular face 1503b. The proximal driving element 802 is constrained to move about the first truncated cone 1503. At least a point on the proximal driving element 802 is secured to the curved face 1503c of the first truncated cone 1503 at the end of the first truncated cone closest to face 1503b. An engagement element 1504 is positioned between the first truncated cone 1503 and a second truncated cone 1505. The second truncated cone 1505 has two parallel planar circular faces 1505a and 1505b and one curved face 1505c. Circular face 1505a has a diameter smaller than that of circular face 1505b. The second truncated cone 1505 is orientated at 180 degrees to the first truncated cone 1503 so as to be in an inverted position relative to the first truncated cone. In other examples, the planar faces of the cones may not be circular, for example they may be generally elliptical. The cone may further be truncated at an angle such that the planar faces of the truncated cone are not parallel. The engagement element 1504 moveably engages face 1503c of the first truncated cone 1503 and face 1505c of the second truncated cone 1505. Faces 1503c and 1505c are parallel to one another at the point at which the engagement element engages them. The engagement element 1504 rotates about an axis 1506. Axis 1506 is parallel to straight lines which entirely intersect faces 1503c and 1505c at the point at which the engagement element engages them. The engagement element has a width equal to the distance between the first and second truncated cones. A distal driving element 806 engages the second truncated cone 1505. The distal driving element 806 is constrained to move about the second truncated cone 1505. At least a point on the distal driving element 806 is secured to the curved face 1505c of the second truncated cone 1505. The proximal and distal driving elements 802 and 806 may be secured to the first and second truncated cones 1503 and 1505, respectively using a bead, pin, clip or other adhesive. Alternatively, the curved surfaces 1503c and 1505c of the first and second truncated cones may comprise one or more grooves. The proximal driving element 802 may sit in a groove of curved surface 1503c of first truncated cone 1503. The distal driving element 806 may sit in a groove of curved surface 1505c of second truncated cone 1505.

In this example, a robot arm (not shown) transfers drive to the end effector of the instrument as follows: movement of a drive assembly interface element (not shown) moves an instrument interface element 801 which moves the proximal driving element 802. Motion of the proximal driving element causes rotation of the first truncated cone 1503. Rotation of the first truncated cone causes rotation of the engagement element 1504 which causes rotation of the second truncated cone 1505. Rotation of the second truncated cone 1505 moves a driving element 806, which moves joint 808 which moves the end effector 807 (seen in FIGS. 8 to 10).

The ratio of the displacement of the proximal driving element 802 l1 to the displacement of the distal driving element 806 l2 depends on the relative rotation of the two truncated cones. The ratio of the displacement of the proximal driving element 802 l1 to the displacement of the distal driving element 806 l2 is therefore a function of:
a) the diameter of the first truncated cone 1503 d1a at the point at which the proximal driving element engages the first truncated cone;
b) the diameter of the second truncated cone 1505 d2a at the point at which the distal driving element engages the second truncated cone;
c) the diameter of the first truncated cone 1503 d1b at the point at which the engagement element 1504 engages the first truncated cone; and
d) the diameter of the second truncated cone 1505 d2b at the point at which the engagement element 1504 engages the second truncated cone.

The ratio of the displacement of the proximal driving element 802 l1 to the displacement of the distal driving element 806 l2 is a function of the ratios:

$$\frac{d 1 a}{d 2 a} and \frac{d 1 b}{d 2 b}$$

The ratio of the displacement of the proximal driving element 802 l1 to the displacement of the distal driving element 806 l2 can be altered by changing the ratio d1a/d2a. The truncated cones 1503 and 1505 may comprise one or more grooves. The proximal driving element 802 may sit in a groove of the first truncated cone 1503. The distal driving element 806 may sit in a groove of the second truncated cone 1505 If each truncated cone comprises a number of grooves, each driving element may be configured to shift between the respective grooves. For example, the positions of the truncated cones may be manually adjusted such that the relative position between each cone and the respective driving element constrained to move around it is altered. The manual mechanism could be a screw that may be tightened. Alternatively, the positions of the cones could be altered using a dedicated servomotor. Due to the change in relative position between a driving element and a truncated cone, the driving element may slide on the cone's surface into different grooves. Therefore, there may be a number of points on the cone (values of h) at which the driving element may engage the cone. Therefore, there may be a discrete number of possible values of d1a and of d2a, and a discrete number of possible ratios d1a/d2a.

The driving elements may be secured to the truncated cones in another way. For example, the driving elements may be secured to the truncated cones using a fixing element such as a bead, clip, pin or using an adhesive. Alternatively, friction between the driving elements and the truncated cones may allow the driving elements to engage the respective truncated cones. The driving elements may be configured to engage the truncated cones at any point on their curved surfaces. The driving element may be configured to engage the curved surfaces at any point along the longitudinal axes of the truncated cones (at any value of h). For example, the proximal driving element 802 could be secured to the first truncated cone 1503 at any value of h. Therefore, there may be a continuous range of possible values of d1a. Similarly, there may be a continuous range of possible values of d2a. In this way, there may be a large continuous range of possible values of d1a/d2a.

The ratio of the displacement of the proximal driving element 802 l1 to the displacement of the distal driving element 806 l2 can be altered by changing the ratio d1b/d2b. Ratio d1b/d2b is calculated from the relative diameters of the first and second truncated cones at the point at which the engagement element 1504 engages both cones. The engagement element may comprise one or more protrusions which mesh with one or more grooves or indents in the truncated cones. For example, the first truncated cone may comprise a number of grooves at different points along its longitudinal axis (different values of h). The engagement element may move from one groove to another. For example, the position of the engagement element may be manually adjusted such that the relative position between the engagement element and each truncated cone is altered. The manual mechanism could be a screw that may be tightened. Alternatively, the position of the engagement element could be altered using a dedicated servomotor. In some examples, the truncated cones may be required to move to allow the engagement to transition from one groove to another. Therefore there may be a discrete number of points at which the engagement element can engage with the first truncated cone, and a discrete number of possible values for d1b. Similarly there may be a discrete number of points at which the engagement element can engage with the second truncated cone, and a discrete number of possible values for d2b. Therefore there may be a discrete number of possible ratios d1b/d2b.

Alternatively, the engagement element may be configured to engage with the truncated cones in another way such that the engagement element can engage with both truncated cones at any point along their respective longitudinal axes. Therefore, there may be a continuous range of values of d1b and d2b and a large continuous range of possible values of d1b/d2b. The engagement element may be configured to move between any two points on the curved surfaces of each of the truncated cones. For example, the engagement element could be a sphere which can rotate to move along the rotation axes of the cones. In an example where the truncated cones do not comprise grooves, the engagement element may engage with the truncated cones due to friction between the engagement element and the cones. The engagement element may be capable of transferring frictional drive in a similar way to a belt.

This gearing mechanism enables the instrument to be highly customisable. The operator of the instrument is able to vary four parameters of the mechanism so as to obtain a desired ratio of the displacement of the proximal driving element 802 l1 to the displacement of the distal driving element 806 l2. In some examples, each of the four parameters may take any value within a continuous range. Therefore, rather than achieving a ratio l1/l2 that is just more suitable for a particular procedure, the operator is able to modify the instrument so as to operate according to a ratio l1/l2 that is equal to the desired ratio.

In the majority of examples, the ratio of the displacement of the proximal driving element 802 l1 to the displacement of the distal driving element 806 l2 depends on all four parameters d1a, d2a, d1b and d2b. However, there are two scenarios, where the ratio l1/l2 is dependent on only two of these parameters. These are:
1) Where the engagement element engages both truncated cones at a point such that the diameters of the two truncated cones at the point at which the engagement element engages them are equal (d1b=d2b). In the example seen in FIG. 15, the cones are the same shape and inverted with respect to one another, so this point is at h/2. When d1b=d2b, the displacement of the proximal driving element l1 which is transferred to the distal driving element l2 depends only on the ratio of the diameter of the first truncated cone 1503 at the point at which the proximal driving element 802 engages the first truncated cone d1a to the diameter of the second truncated cone 1505 at the point at which the distal driving element 806 engages the second truncated cone d2a (d1a/d2a).
2) Where the diameter of the first truncated cone 1503 at the point at which the proximal driving element 802 engages the first truncated cone d1a is equal to the diameter of the second truncated cone 1505 at the point at which the distal driving element 806 engages the second truncated cone d2a (d1a=d2a). In this example, the displacement of the proximal driving element l1 which is transferred to the distal driving element l2 depends only on the ratio of the diameters of the first and second truncated cones at the point at which the engagement element engages both truncated cones (d1b/d2b).

The robot arm 102 comprises motors (not shown) to allow the arm to operate in the manner described herein. i.e. motors in the arm cause the drive assembly to transfer drive to the instrument as previously described. Controllers for the motors are distributed within the robot arm. As seen in FIG. 2, the surgical robot 100 forms part of a system also including a surgeon command interface 201 and a control unit 202. The control unit comprises a processor 203 and a memory 204. The memory 204 stores in a non-transient way software that is executable by the processor to control the operation of the motors to cause the arm 102 to operate in the way described. The software can control the processor 203 to cause the motors to drive in dependence on inputs from the surgeon command interface. The control unit 202 is coupled to the motors for driving them in accordance with outputs generated by execution of the software. The surgeon command interface 201 comprises one or more input devices whereby a user can request motion of the end effector in a desired way. The input devices could, for example, be manually operable mechanical input devices such as control handles or joysticks, or contactless input devices such as optical gesture sensors. The software stored in memory 204 is configured to respond to those inputs and the processor is configured to execute the software to cause the joints of the arm and instrument to move accordingly. In summary, a surgeon at the command interface 201 can control the instrument 103 to move in such a way as to perform a desired surgical procedure. The control unit 202 and/or the command interface 201 may be remote from the arm 102.

In order for the desired surgical procedure to be performed in the desired way, the control unit must take into account a number of pieces of information about the robot arm 102 and the instrument 103. Pieces of information about the instrument may include for example, the type of instrument and the positions of components of the end effector. The instrument 103 may comprise a processor and a transmitter and be configured to transmit information to the control unit 202. The instrument may also comprise a memory configured to store information about the instrument. Information transmitted to the control unit may further include the ratio of the displacement of the proximal driving element 802 l1 to the displacement of the distal driving element 806 l2. For example, the instrument may store in memory the ratio l1/l2.

In an example such as that shown in FIG. 8, the instrument comprises a single pulley 805 about which the distal driving element is configured to move. Such an instrument may thus only operate according to one known ratio l1/l2. This instrument may therefore permanently store the value of l1/l2 and transmit it to the control unit to assist the control unit with controlling the surgical robot to perform the desired surgical procedure.

In another example, such as one similar to that shown in FIG. 13, the ratio of the displacement of the proximal driving element 802 l1 to the displacement of the distal driving element 806 l2 may not be known. In this example, the instrument may be configured to detect one or both of the displacements l1 and l2. The instrument may comprise one or more sensors. The instrument may comprise a sensor for detecting the displacement of the distal driving element 806 or the displacement of the end effector 807. The instrument may comprise a sensor for detecting the displacement of the instrument interface element 801 or the proximal driving element 802. Detecting a displacement may be performed in a number of ways, for example by measuring a tension of the driving element or using a visual sensor.

Alternatively, the control unit may be configured to calculate the ratio l1/l2. The control unit may receive information from the instrument and use the information to calculate the ratio. For example, the instrument may transmit to the control unit, the diameters of the first and second pulleys d1 and d2. In the example seen in FIG. 13 in which the distal driving element may be constrained by one of three possible pulleys, the instrument may be configured to detect which pulley the distal driving element is constrained to move about and transmit that information to the control unit. Similarly, the instrument may be configured to detect the diameter of the pulley about which the distal driving element is constrained to move and transmit that information to the control unit. Each pulley may comprise a sensor, the sensor being configured to detect whether a driving element is constrained to move about that pulley. The instrument may be configured to transmit the output of the sensors to the control unit.

As previously mentioned, the displacement of the distal driving element l2 may be detected by the instrument and transmitted to the control unit, but the displacement of the proximal driving element l1 may not be known. The control unit may be configured to derive the value of the displacement of the proximal driving element l1. As described above, the control unit instructs motors in the arm to actuate movement of the arm. The magnitude of the force imparted on the drive assembly interface element e.g. 403 may be measured by a force sensor in the arm and the value of the force transmitted to the control unit. The control unit may thus be configured to use the force applied to the drive assembly interface element to derive the displacement of the end of the proximal driving element. The arm may alternatively or additionally comprise a motion sensor configured to measure the displacement of the drive assembly interface element 403, which is equal to the displacement of the end of the proximal driving element, l1. In this way the control unit may be configured to derive the displacement of the end of the proximal driving element l1 from a sensed displacement of the drive assembly interface element. The value of l1 may then be transmitted to the control unit. The control unit may then calculate the ratio l1/l2.

The ratio l1/l2 or d1/d2 may also be approximated by comparing the tension of the proximal driving element to that of the distal driving element. The ratio may also be determined by comparing the desired torque or force at the end effector to the actual force or torque achieved by the end effector. The time taken for the system to achieve the desired force or torque may also provide an indication of the current ratio between the diameter of the first pulley and the diameter of the second pulley.

It is desirable that the control unit used to control the surgical robot to perform the desired surgical procedure either receives or determines the proportion of the force imparted on the instrument interface element that is transferred to the end effector. Particularly in an instrument such as that illustrated in FIG. 13, in which multiple possible ratios l1/l2 are enabled, it is advantageous that the control unit is configured to receive and/or calculate this information so that it can provide suitable driving signals to the robot so as to cause the robot to perform the desired surgical procedure safely and effectively.

The examples described herein have been explained in relation to a driving mechanism comprising one drive assembly interface element, one instrument interface element, one gearing mechanism, one joint and one end effector, however it will be appreciated that the same principles can be extended to a plurality of each of these, for example using the drive assembly interface and instrument interface shown in FIGS. 4 and 6. One or more end effectors may be actuated about multiple joints simultaneously using a number of independent gearing mechanisms. It will be appreciated that a single instrument may comprise more than one gearing mechanism and may comprise a combination of different gearing mechanisms such as any combination of those shown in FIGS. 8 to 14.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A robotic surgical instrument comprising:
a shaft;
an articulation attached to a distal end of the shaft, the articulation configured to articulate an end effector, the articulation driveable by a distal driving element; and
a driving mechanism comprising:
an instrument interface element secured to an end of a proximal driving element and configured to engage a drive interface element of a drive assembly, wherein motion of the drive interface element results in a first displacement of the end of the proximal driving element; and
a gearing mechanism engaging the proximal driving element and the distal driving element and being configured to transfer the first displacement of the end of the proximal driving element to a different second displacement of an end of the distal driving element, the gearing mechanism comprising:
a first pulley about which the proximal driving element is constrained to move, the first pulley being configured to rotate about an axis, and having a first pulley radius; and
a second pulley about which the distal driving element is constrained to move, the second pulley being one of a plurality of pulleys each having a different radius and being configured to rotate about the same axis;
where the first pulley radius is different to each of the respective radii of the plurality of pulleys;
in which a ratio of the first and second displacements is a function of the ratio of the first pulley radius to the radius of the second pulley.

2. (canceled)

3. (canceled)

4. The instrument of claim 1, wherein the distal driving element is constrained to move about one of the plurality of pulleys and the ratio of the first and second displacements is a function of the ratio of the radius of the first pulley to the radius of the pulley about which the distal driving element is constrained to move.

5. The instrument of claim 4, wherein the ratio of the first and second displacements is selected from a discrete number of ratios.

6. (canceled)

7. The instrument of claim 1, wherein the gearing mechanism comprises a toothed rack, the first pulley comprises a toothed gear and is configured to engage the toothed rack such that motion of the toothed rack results in rotation of the toothed gear.

8. The instrument of claim 7, wherein the ratio of the first and second displacements is a function of the dimensions of the toothed rack and toothed gear, and the radius of the second pulley.

9. The instrument of claim 7, wherein the proximal driving element further comprises
a first rod secured to the instrument interface element; and
a second rod secured to the toothed rack and configured to moveably engage with the first rod such that displacement of the first rod results in displacement of the toothed rack, wherein the ratio of the first and second displacements is a function of the dimensions of the toothed rack, the toothed gear, the first rod and the second rod.

10. The instrument of claim 9, wherein the first rod comprises an aperture and the second rod is configured to be threaded through the aperture in the first rod.

11. The instrument of claim 1, wherein the first pulley is a first truncated cone and the second pulley is a second truncated cone.

12. (canceled)

13. (canceled)

14. (canceled)

15. (canceled)

16. The instrument of claim 1, the instrument comprising a memory and being configured to store in memory the ratio of the first and second displacements.

17. The instrument of claim 16, the instrument being configured to transmit the ratio of the first and second displacements to a control unit configured to control a surgical robot.

18. A system comprising:
a robot arm;
the instrument of claim 1; and
a control unit being configured to determine the ratio of the first and second displacements.

19. The system of claim 18, wherein the control unit is configured to determine the ratio of the first and second displacements using information transmitted from the instrument to the control unit.

20. The system of claim 18, wherein the control unit is configured to determine the second displacement by measuring the tension in the distal driving element and/or by measuring motion of the end effector of the instrument.

21. The system of claim 18, the robot arm comprising a drive assembly having a drive assembly interface element, the drive assembly interface element being configured to engage with the instrument interface element such that motion of the drive interface element results in motion of the instrument interface element; and the robot arm being configured to apply a force to the drive assembly interface element, wherein the control unit is configured to derive the first displacement from a sensed displacement of the drive assembly interface element.

22. The system of claim 18, wherein each pulley in the plurality of pulleys of the instrument comprises a sensor configured to detect whether the distal driving element is constrained to move about that pulley, and the instrument is configured to communicate to the control unit, about which pulley of the plurality of pulleys the distal driving element is constrained to move.

23. The system of claim 22, wherein the instrument is configured to communicate to the control unit, the diameter of the pulley about which the distal driving element is constrained to move.

24. The system of claim 23, wherein the control unit is configured to determine the ratio of the first and second displacements using the diameter of the first pulley and the diameter of the pulley about which the distal driving element is constrained to move.

25. A robotic surgical instrument comprising:
a shaft;
an articulation attached to a distal end of the shaft, the articulation configured to articulate an end effector, the articulation driveable by a distal driving element; and
a driving mechanism comprising:
an instrument interface element secured to an end of a proximal driving element and configured to engage a drive interface element of a drive assembly, wherein motion of the drive interface element results in a first displacement of the end of the proximal driving element; and
a gearing mechanism engaging the proximal driving element and the distal driving element and being configured to transfer the first displacement of the end of the proximal driving element to a different second displacement of an end of the distal driving element, wherein the gearing mechanism comprises:
a first truncated cone about which the proximal driving element is constrained to move;
a second truncated cone about which the distal driving element is constrained to move; and
an engagement element configured to moveably engage with the first truncated cone and the second truncated cone so as to transfer rotation of the first truncated cone to rotation of the second truncated cone.

26. The instrument of claim 25, wherein a ratio of the first and second displacements is a function of the radius of the first truncated cone at the point at which the proximal driving element is constrained, to the radius of the second truncated cone at the point at which the distal driving element is constrained.

27. The instrument of claim 25, wherein a ratio of the first and second displacements is a function of the radius of the first truncated cone at the point at which the engagement element engages the first truncated cone, to the radius of the second truncated cone at the point at which the engagement element engages the second truncated cone.
